(19) United States
(12) Patent Application Publication
(10) Pub. No.: US 2026/0237461 A1
(43) Pub. Date: Aug. 13, 2026

(54) DETECTING VIRAL SEQUENCES IN METAGENOME DATA

(71) Applicant: Sanofi, PARIS (FR)

(72) Inventors: Sourav Roy Choudhury, Waban, MA (US); Youngji Na, Natick, MA (US); Eugenia Lyashenko, Boston, MA (US)

(21) Appl. No.: 19/150,653

(22) PCT Filed: Jan. 23, 2024
(86) PCT No.: PCT/US2024/012590

§ 371 Date: Jul. 24, 2025

Related U.S. Application Data
(60) Provisional application No. 63/481,466, filed on Jan. 25, 2023.

(30) Foreign Application Priority Data
May 3, 2023 (EP) 23315155.4

(51) Int. Cl. G16B 30/10; G16B 30/20

(52) U.S. Cl. CPC G16B 30/10; G16B 30/20

(57) ABSTRACT

Provided herein are methods and systems for detecting polynucleotide sequences encoding viral capsids in metagenome data. The methods and systems disclosed herein can include a sequence alignment-based module, a gene-based data processing module, and further characterization of putative viral sequences to identify novel polynucleotide sequences encoding viral capsids in metagenome data.

### CROSS-REFERENCE TO RELATED APPLICATIONS

This application claims the benefit of U.S. Provisional Patent Application No. 63/481,466, filed Jan. 25, 2023, and European Patent Application No. 23315155.4, filed May 3, 2023, the contents of which are incorporated herein by reference in their entirety.

### TECHNICAL FIELD

This disclosure relates to methods and systems for detecting polynucleotide sequences encoding viral capsids in metagenome data.

### BACKGROUND

At least hundreds of millions of virus species are likely to exist on Earth, but sequences for only tens of thousands of virus species are deposited in the central GenBank virus database. Mining the virome has revealed thousands of novel viruses, with the rate of virus discovery increasing exponentially and driven largely by the increased availability of high-throughput sequencing. Previously, researchers have used metagenomics approaches to identify and characterize bacterial populations. However, it has become apparent that such profiling can also detect viral sequence.

Naturally occurring novel AAV capsids can be detected in metagenomic data. Indeed, there has been a recent acceleration of mining viruses in metagenome samples as a result of increasing data availability and decrease in computing costs and time. Over 160 currently known AAV isolates have been identified from genomic samples from human or non-human primate (NHP) sources. The AAVs are classified into six genetic groups (clades A-F) and two clonal isolates (AAV4 and AAV5) based on phylogeny and serology.

Computational approaches to identify viral sequences from metagenome data can be grouped into four broad categories based on their approaches. First, sequence alignment-based approaches, wherein viral metagenomic reads are determined based on sequence alignment. However, the current virus genome databases are biased towards certain types of viruses whose hosts are cultivable in the lab, and the current virus references are an incomplete representation of the entirety of viral diversity. Second are gene-based similarity approaches, wherein viral sequences are detected by comparing genes in the query sequence against viral gene databases. However, the gene-based similarity methods require query sequences to contain complete genes, so they are not able to predict sequences from non-coding regions. Third are genomic feature-based machine learning (ML) approaches, wherein genomic features are used to distinguish viral from microbial sequences as inputs to train (ML) classifiers. However, the manual curation and inspection involved in these ML approaches is often a critical step in the process to detect genomic features. Fourth are k-mer frequency-based ML approaches, wherein the frequency of DNA “words” of length k found in known viral genomes are provided as inputs to train ML classifiers. However, it is known that viral sequences can display homology to their host genomic sequences, perhaps as an evolutionary strategy in order to overcome host genome defenses, and this phenomenon of virus-host genome homology can cause classifiers based on k-mer frequencies to have poor performance in terms of overall accuracy and recall. Furthermore, although several tools have previously been developed to detect virus sequences in metagenome data, the pipelines are not specialized for AAV capsids, which can be particularly useful for applications including gene therapy in human.

Therefore, there exists a need for methods for detection and identification of viral sequences encoding novel viral capsids, e.g., adeno-associated virus (AAV) capsids, in metagenome data with increased sensitivity for rare sequences, increased computing speeds, and decreased computational costs.

### SUMMARY

The present disclosure is based, in part, on the discovery that based on the similarity with naturally isolated AAVs as references, novel AAV isolates can be identified in publicly available metagenome data. Based on the identified sequence, functional properties for the virus, e.g. of the capsid, are dependent on the AAV capsid amino acid sequence, capsid structure, and interactions with host cell factors. The present disclosure is directed towards methods, systems, and computer programs for identification of novel AAV capsids in metagenome data. Provided herein are methods to mine, for example next-generation sequencing (NGS) data, for putative Parvoviridae contigs, further extract sequences encoding AAV-like capsid proteins from identified contigs, and assess the novelty of identified sequences.

In one aspect, provided herein are methods for identifying novel viral polypeptide sequences by screening polynucleotide sequencing data. The methods provided herein can include providing polynucleotide sequencing data including a plurality of sequencing reads; aligning each of the plurality of sequencing reads to one or more reference sequences; assembling, from the plurality of sequencing reads aligned to the one or more reference sequences, a plurality of sequence contigs; selecting, based on the one or more reference sequences, one or more contigs comprising a viral open reading frame (ORF) sequence; and identifying, based on a translated polypeptide sequence of the viral ORF, one or more novel viral polypeptide sequences. In some embodiments, the one or more reference sequences are virus references sequences. In some embodiments, the one or more virus reference sequences are Parvoviridae reference sequences. In some embodiments, the one or more virus reference sequences are adeno-associated virus (AAV) reference sequences. In some embodiments, the AAV reference sequences are sequences encoding an AAV cap protein. In some embodiments, the polynucleotide sequencing data are metagenome data. In some embodiments, the plurality of sequencing reads are generated by next-generation sequencing (NGS).

In some embodiments, the selecting step further include prescribing a minimum circular contig length. In some embodiments, the minimum circular contig length is 4000 bp. In some embodiments, the selecting step further includes prescribing a minimum linear contig length. In some embodiments, the minimum linear contig length is 1500 bp. In some embodiments, the minimum circular contig length is 4000 bp and the minimum linear contig length is 1500 bp. In some embodiments, the identifying step further includes prescribing a minimum ORF length. In some embodiments, the minimum ORF length is 685 amino acids. In some embodiments, the identifying step further includes BLAST alignment analysis. In some embodiments, BLAST alignment analysis is performed against previously annotated natural AAV isolates and/or previously discovered Parvoviridae amino acid sequences.

In another aspect, provided herein is a computer program product tangibly embodied on a computer-readable medium, including instructions, that when executed by one or more processors, are configured to receive polynucleotide sequencing data including a plurality of sequencing reads; align each of the plurality of sequencing reads to one or more reference sequences; assemble, from the plurality of sequencing reads aligned to the one or more reference sequences, a plurality of sequence contigs; select, based on the one or more reference sequences, one or more contigs comprising a viral open reading frame (ORF) sequence; and identify, based on a translated polypeptide sequence of the viral ORF, one or more novel viral polypeptide sequences. In some embodiments, the one or more reference sequences are virus references sequences. In some embodiments, the one or more virus references are Parvoviridae sequences. In some embodiments, the virus reference sequences are adeno-associated virus (AAV) reference sequences. In some embodiments, the AAV reference sequences are sequences encoding an AAV cap protein. In some embodiments, the polynucleotide sequencing data are metagenome data. In some embodiments, the plurality of sequencing reads are generated by next-generation sequencing (NGS).

In some embodiments, the selecting step further includes prescribing a minimum circular contig length. In some embodiments, the minimum circular contig length is 4000 bp. In some embodiments, the selecting step further includes prescribing a minimum linear contig length. In some embodiments, the minimum linear contig length is 1500 bp. In some embodiments, the minimum circular contig length is 4000 bp and the minimum linear contig length is 1500 bp. In some embodiments, the identifying step further includes prescribing a minimum ORF length. In some embodiments, the minimum ORF length is 685 amino acids. In some embodiments, the identifying step further includes BLAST alignment analysis. In some embodiments, BLAST alignment analysis is performed against previously annotated natural AAV isolates and/or previously discovered Parvoviridae amino acid sequences.

The present disclosure provides several advantages. Provided herein is a custom, AAV-specific pipeline that extracts and identifies novel AAVs in putative Parvoviridae containing contigs. The methods and systems are specialized for AAVs and combine two computational pipelines to achieve computation speeds up to 500-fold faster than previously available. These advantages allow precise characterization of relatively rare novel AAVs in petabytes of metagenome data, thereby expanding the repertoire of AAV isolates for human gene therapies.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram generally illustrating the computational workflow disclosed herein. The workflow can include (1) processing millions of sequencing read archive (SRA) sequencing reads; (2) filtering for metagenome sequencing reads; (3) filtering for virus-derived sequencing reads; (4) sequence-based alignments to identify parvoviral-derived sequencing reads; and (5) identifying AAV-derived sequencing reads.

FIG. 2 is a flowchart summarizing post hoc analysis steps for characterization of putative AAV cap ORFs to confirm novelty.

FIG. 3 is a phylogenetic tree of previously known AAV clades. The computational pipeline disclosed herein identified 44 novel AAV capsids, a subset of which form three putative undiscovered AAV branches, as indicated on the phylogenetic tree diagram.

FIG. 4 is a phylogenetic tree diagram showing a subset of 163 isolates of AAV viruses, highlighting nine contigs close to AAV2 (Clade B) that were identified by the computational pipeline disclosed herein as complete and novel.

FIG. 5 is a diagram of computer system components that can be used to implement methods for detecting polynucleotide sequences encoding viral capsids in metagenome data.

FIG. 6 is a multiple sequence alignment (MSA) showing partial amino acid sequences of nine putative novel AAV cap ORFs identified by the computational methods and systems disclosed herein aligned against the AAV2 VP1 reference sequence. Divergent amino acid residues are highlighted by gray bars.

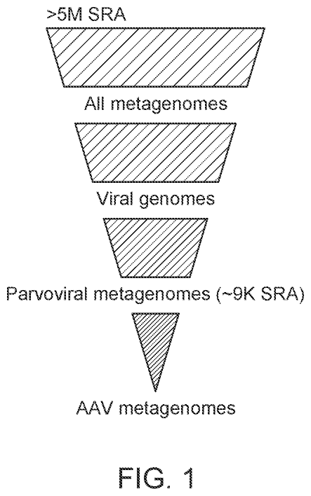

### DETAILED DESCRIPTION

### Adeno-Associated Virus

Adeno-associated viruses (AAV) are small viruses that infect humans and some other primate species. They belong to the genus Dependoparvovirus, which in turn belongs to the family Parvoviridae. They are small, approximately 26 nm in diameter, replication-defective, non-enveloped viruses and have linear single-stranded DNA (ssDNA) genome of approximately 4.8 kilobases (kb). The genome comprises inverted terminal repeats (ITRs) at both ends of the DNA strand, and two open reading frames (ORFs): rep and cap. The rep ORF is composed of four overlapping genes encoding Rep proteins required for the AAV life cycle, and the latter contains overlapping nucleotide sequences of capsid proteins: VP1, VP2 and VP3, which interact to form a capsid with icosahedral symmetry.

At the 5′ end of the AAV genome there are two promoters called p5 and p19, from which two overlapping messenger ribonucleic acids (mRNAs) of different length can be produced. Each of these contains an intron which can be either spliced out or not. Given these possibilities, four various mRNAs, and consequently four various Rep proteins with overlapping sequence can be synthesized. Their names depict their sizes in kilodaltons (kDa): Rep78, Rep68, Rep52 and Rep40. Rep78 and 68 can specifically bind the hairpin formed by the ITR in the self-priming act and cleave at a specific region, designated terminal resolution site, within the hairpin. The Rep proteins are also necessary for the AAVS1-specific integration of the AAV genome. All four Rep proteins bind ATP and possess helicase activity.

The right side of a positive-sensed AAV genome encodes overlapping sequences of three capsid proteins, VP1, VP2 and VP3, which originate from one promoter, designated p40. The molecular weights of these proteins are 87, 72 and 62 kD, respectively. The AAV capsid is composed of a mixture of VP1, VP2, and VP3 totaling 60 monomers arranged in icosahedral symmetry in a ratio of 1:1:10, with an empty mass of approximately 3.8 MDa. The cap gene produces an additional, non-structural protein called the Assembly-Activating Protein (AAP). This protein is produced from ORF2 and is essential for the capsid-assembly process.

All three VPs are translated from one mRNA. After this mRNA is synthesized, it can be spliced in two different manners: either a longer or shorter intron can be excised resulting in the formation of two pools of mRNAs: a 2.3 kb- and a 2.6 kb-long mRNA pool. Usually, especially in the presence of adenovirus, the longer intron is preferred, so the 2.3-kb-long mRNA represents the so-called “major splice”. In this form the first AUG codon, from which the synthesis of VP1 protein starts, is cut out, resulting in a reduced overall level of VP1 protein synthesis. The first AUG codon that remains in the major splice is the initiation codon for VP3 protein. However, upstream of that codon in the same open reading frame lies an ACG sequence (encoding threonine) which is surrounded by an optimal Kozak context. This contributes to a low level of synthesis of VP2 protein, which is actually VP3 protein with additional N terminal residues, as is VP1.

AAV possesses unique features that make it attractive as a vector for delivering foreign DNA to cells, for example, in gene therapy. AAV infection of cells in culture is non-cytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

### Computational Workflow

Provided herein are systems and methods for detecting polynucleotide sequences encoding viral capsids in metagenome data. In some embodiments, the systems and methods include a computational workflow for processing NGS data generated from sequencing of metagenomics samples. In some embodiments, the viral capsids are encoded by sequences characteristic of Parvoviridae viruses. In some embodiments, the viral capsids are encoded by sequences characteristic of AAV. In some embodiments, the computational workflow disclosed herein begins with a sequence alignment-based approach to screen metagenome data comprising NGS sequencing reads for signatures of Parvoviridae polynucleotide sequences. In some embodiments, a sequence alignment-based approach is used to select for NGS reads comprising signatures of Parvoviridae polynucleotide sequences. In some embodiments, select NGS reads where Parvovirus reads were found are further processed by a gene-based data processing modules, wherein highly sensitive models of hallmark viral genes, e.g. AAV cap genes, are used to discover familiar or divergent viral sequences from user-input contigs. In some embodiments, after the viral gene-based data processing module is used, Parvovirus contigs are prioritized in further post hoc analysis based on additional criteria, for example, presence of AAV hallmark genes and/or BLAST alignments to known AAV-like genes. In some embodiments, after prioritizing Parvovirus contigs, cap gene open reading frames (ORF) are identified. In some embodiments, in order to identify AAV-like genome sequences, cap open reading frames are selected based on criteria including, for example, length of open reading frame, presence of start codon and presence of stop codon. In some embodiments in order to identify Parvoviridae contigs similar to previously discovered naturally isolated AAVs, phylogenetic tree analysis is performed. In some embodiments, in order to determine novelty of the identified candidate sequences, candidate sequences are aligned using BLAST analysis against various databases of known AAV sequences. Examples of databases of known AAV sequences include the NCBI VIRUS database, the NCBI NR (non-redundant protein) database, and the NCBI PATAA (patented protein sequence) database. In some embodiments, after determining novelty of the identified candidate sequences encoding novel AAV capsids, sequences encoding novel AAV capsids are curated based on the meta data of the sequence read archive (SRA) in which the sequence encoding the novel AAV was identified in order to remove artifacts or false positives.

Disclosed herein is a computational pipeline for identification of novel AAV capsids in a pool of candidate Parvoviridae contigs. The pipeline disclosed herein can include parameters including AAV capsid protein length, a filter to extract all ORFs in a candidate contigs, and a filter to retrieve only complete ORFs (starting with M (start codon, methionine) and ending with a stop codon). This has not been previously articulated for the search of AAV capsids specifically. In some embodiments, the pipeline further includes BLAST searches to determine if the putative novel AAV cap sequence has been detected in any of the databases or in patent databases. In some embodiments, the pipeline further includes placing the candidate sequence encoding the complete AAV cap on the phylogenetic tree of known 160 natural AAV isolates.

### Sequence Alignment

In some embodiments, the computational methods disclosed herein include a sequence alignment-based module to screen metagenome data comprising NGS sequencing reads for signatures of Parvoviridae polynucleotide sequences. In some embodiments, the computational pipeline begins with this sequence alignment-based approach. In some embodiments, this aspect of the computational method relies on mapping NGS sample reads to a reference pan-viral genome. In some embodiments, samples with any reads mapped to AAV genomes/genes present are identified using a sequence alignment-based approach.

In some embodiments, the computation workflow begins with the sequence alignment-based module to screen metagenome data comprising NGS sequencing reads for signatures of Parvoviridae polynucleotide sequences to pre-screen the publicly available SRA for NGS reads comprising Parvoviridae sequences. This pre-screening step advantageously reduces computation time by decreasing the number of metagenome-derived NGS reads needed to be screened by downstream modules to identify novel AAV-associated sequences. Downstream steps of the pipeline, e.g., a gene-based similarity approach, can be computationally intensive due to the de novo contig assembly steps involved. This is a computationally intensive step for AAV-associated sequences, as by nature, these viruses are relatively rare in metagenome data, e.g., <0.2%, and therefore, the frequency of novel previously unannotated AAVs in metagenome datasets is even rarer. For example, for the previously published Serratus work (Edgar RC, et al. Petabase-scale sequence alignment catalyses viral discovery. Nature. 2022 February; 602(7895): 142-147, which is herein incorporated by reference in its entirety), among the publicly available SRA datasets, approximately 9,000 SRA comprised Parvoviridae sequences reads (Serratus score 20-100, where 100 is the maximum) out of approximately 5,800,000 SRA, i.e., approximately 0.15% of reads. As another example, Hsu et al. profiled approximately 800 human tissue biopsies with long read sequencing and found 1 novel AAV-associated sequence, i.e., approximately 0.125%. These previously published results suggest that detecting AAVs with only a de novo contig assembly pipeline for a random pool of SRAs would require screening millions of SRA with potentially prohibitively high computing costs.

In some embodiments, the Serratus cloud computing infrastructure is used to screen metagenome data comprising NGS sequencing reads for signatures of Parvoviridae polynucleotide sequences (Edgar RC, et al. Petabase-scale sequence alignment catalyses viral discovery. Nature. 2022 February; 602(7895): 142-147, which is herein incorporated by reference in its entirety). Serratus is a bioinformatics platform that screens sequences from the NCBI SRA (National Center for Biotechnology Information Sequence Read Archive) for similarity to known viruses (alignment-based approach). The published Serratus project analyzed 5.7 million biologically diverse samples (10.2 petabases). In some embodiments, in order to reduce the test sample size for the computational pipeline disclosed herein, a computation tool, e.g., Serratus, is used to extract samples where Parvoviridae reads are identified in publicly available large-scale metagenome data.

### Gene-Based Similarity Approach

In some embodiments, the computational methods disclosed herein further include a gene-based data processing module, wherein highly sensitive models of hallmark viral genes, e.g. AAV cap genes, are used to discover familiar or divergent viral sequences from user-input contigs. For example, contigs comprising NGS reads where Parvovirus reads were identified by the sequence alignment-based module can be further processed by the gene-based data processing module in order to identify and select NGS reads that are likely to comprise novel AAV capsid genes. In some embodiments, the gene-based data processing module is Cenote-Taker2 (Tisza MJ, et al. Cenote-Taker 2 democratizes virus discovery and sequence annotation. Virus Evol. 2020 Dec. 30; 7(1): veaa100, which is hereby incorporated by reference in its entirety). Cenote-Taker2 is a virus discovery and annotation tool that utilizes highly sensitive models of hallmark virus genes to discover familiar or divergent viral sequences from user-input contigs i.e., an assembly-based approach). In some embodiments, in order to identify all possible putative viruses among metagenome NGS reads, Cenote-Taker2 is used to identify and select NGS reads comprising genes encoding AAV capsids.

In some embodiments, functionalities and parameters of the gene-based data processing module, e.g., Cenote-Taker2, are customized to promote the identification of NGS reads comprising genes encoding AAV capsids and to enhance computational efficiency. In some embodiments, these parameters can include, for example, minimum linear contig length, minimum circular contig length, and minimum number of hallmark genes.

The length of AAV2 wild type complete genome (GenBank accession: AF043303.1) is approximately 4.7 kbp. The length of complete VP 1, the longest cap ORF, is 2205 bp. By default, Cenote-Taker 2 code published in GitHub sets both parameters (minimum linear contig length and minimum circular contig length) to be 1000 bp. This setting results in identifying and abundance of multiple short contigs including phages, non-parvoviruses, or sequences encoding incomplete AAV cap genes. Including these superfluous results significantly extends computation time and increases computation costs.

In some embodiments, in order to reduce computation time and enhance computational efficiency of the gene-based data processing module of the computational pipeline for identifying NGS reads comprising genes encoding AAV capsids, minimum circular contig length is set to 2000 bp, 2500 bp, 3000 bp, 3500 bp, 4000 bp, 4500 bp, or 5000 bp. In some embodiments, minimum circular contig length is set to 2000 bp, 2500 bp, 3000 bp, 3500 bp, 4000 bp, 4500 bp, or 5000 bp while minimum linear contig length is maintained at 1500 bp. In a preferred embodiment, minimum circular contig length is set to 4000 bp, minimum linear contig length is set to 1500 bp, and number of hallmark genes is set to 0. These parameters provide enhanced efficiency for eliminating contigs comprising phage sequences and sequences encoding incomplete AAV cap genes, while reducing computation time and computation costs. Computational resources can be allocated toward post hoc analysis of identified sequences encoding complete AAV cap genes by removing multiple short contigs comprising phage sequences, non-parvovirus sequences, or sequences encoding incomplete AAV cap genes.

In some embodiments, in order to identify contigs comprising NGS reads representing genes encoding novel AAV capsids from the output of the gene-based data processing module, additional criteria are utilized. The criteria can include, for example, presence of relevant hallmark genes. In some embodiments, the relevant hallmark genes for identifying genes encoding novel AAV capsids from the output of the gene-based data processing module can include long cap (VP1), short cap (VP2 or 3), or non-structural AAV proteins. In some embodiments, non-structural AAV proteins can include PDB:3KIC_N-Capsid-protein-Parvo and/or CDD:pfam00740-minor-capsid-protein.

In some embodiments, as a component of the gene-based data processing module, BLAST alignment is used to match putative contigs to AAV-like genes using a publicly available viral reference database. In some embodiments, putative contigs are aligned against a database of reference AAV cap ORFs using BLAST top amino acid sequence similarity to proteins annotated with an “Adeno-Associated”description. This BLAST alignment step can advantageously increase the specificity of the gene-based data processing module for identifying genes encoding novel AAV capsids among contigs that may include genes encoding capsid sequences for other Parvoviridae species. Additionally, some contigs may comprise putative AAV cap ORFs that do not closely match the previously designated AAV cap hallmark gene, but a BLAST alignment with customized parameters can identify a similarity to the AAV cap hallmark gene.

In some embodiments, the gene-based data processing module, e.g., Cenote-Taker2 provides intermediate files as well as a final contig summary file. Based on the intermediate files, annotations and sequences of the contigs in the summary file can be extracted. In some embodiments, steps to extract putative AAV cap ORFs include: 1) acquire contig list from the final contig summary file of the gene-based data processing module output; (2) acquire annotations of the contig list of step (1), wherein annotations can include genomic coordinates of the translated ORFs, protein ID numbers, and protein product name. In some embodiments, based on a string-match of “cap/cap” in the annotation file, protein product names and genomic coordinates of the translated putative cap ORFs are extracted; and (3) acquire polypeptide sequences of putative AAV cap ORFs.

### Post Hoc Analysis: Identification of Novel Complete AAV Caps

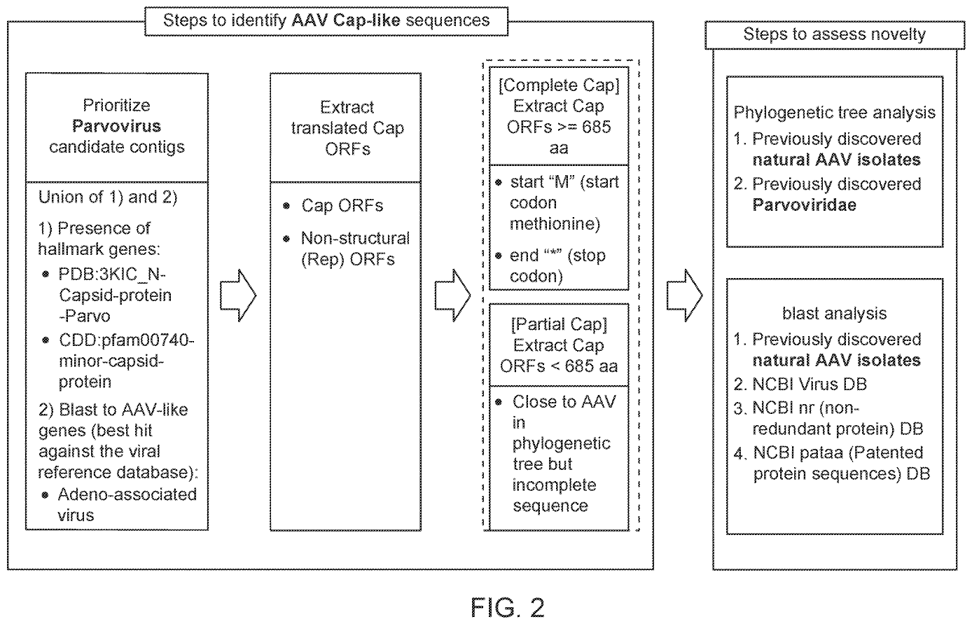

In some embodiments, the computational workflow includes prioritization of contigs including complete putative AAV cap ORFs and includes further post hoc characterization of putative AAV cap ORF to confirm novelty. A flowchart summarizing these post hoc analysis steps is provided in FIG. 2. Factors contributing to the prioritization of contigs including complete putative AAV cap ORFs can include presence of previously annotated AAV hallmark genes. The previously annotated AAV hallmark genes can include, for example, PDB:3KIC_N-Capsid-protein-Parvo and/or CDD:pfam00740-minor-capsid-protein. Additional factors contributing to the prioritization of contigs can include length of the putative AAV cap ORFs. For example, putative ORFs can be extracted filtering for ORFs with a length greater than 500, 550, 600, 650, or 700 amino acids. In a preferred embodiment, putative ORFs with a length of greater than 685 amino acids are extracted. In some embodiments, putative ORFs are filtered for ORFs beginning with a start codon, i.e., a codon encoding a methionine amino acid residue. In some embodiments, putative ORFs are filtered for ORFs ending with a stop codon.

In some embodiments, factors for evaluating novelty of identified putative AAV cap ORFs include phylogenetic tree analysis. In some embodiments the phylogenetic tree analysis include previously annotated natural AAV isolates and/or previously discovered Parvoviridae amino acid sequences. In some embodiments, factors for assessing novelty of identified AAV sequences include BLAST alignment analysis against previously annotated natural AAV isolates and/or previously discovered Parvoviridae amino acid sequences. In some embodiments the BLAST analysis includes the NCBI Virus database; NCBI RefSeq Non-Redundant Protein database; and/or NCBI PATAA patent protein sequence database.

### Computer Implementation of the Methods

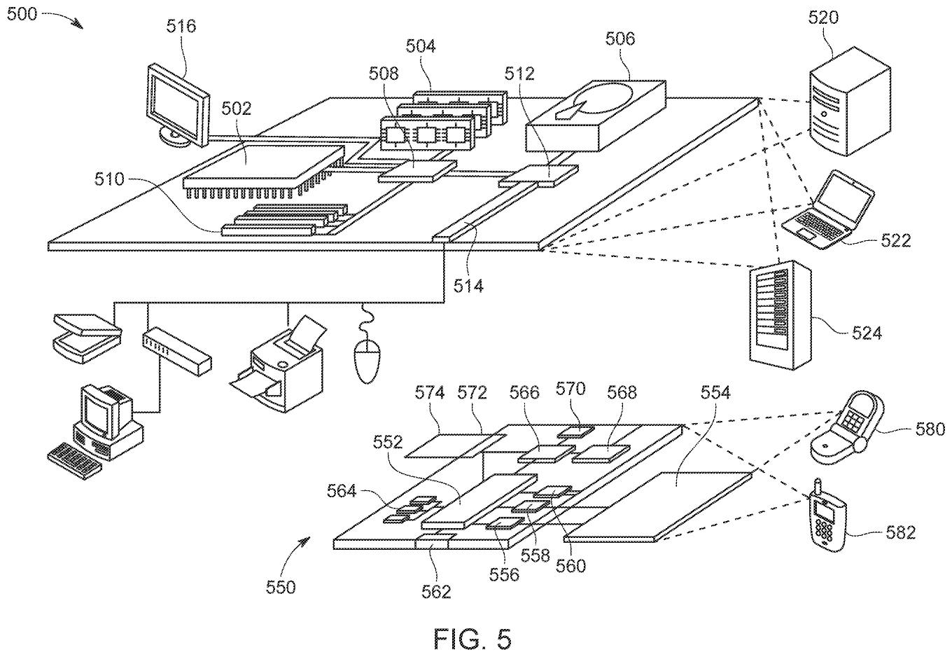

FIG. 5 is a diagram of computer system 500 components that can be used to implement the sequence alignment-based module, gene-based data processing module, and/or the prioritization of contigs including complete putative AAV cap ORFs and further post hoc characterization of putative AAV cap ORF to confirm novelty. The components of computer system 500 can perform the steps of (1) the sequence alignment-based module, (2) gene-based data processing module, and/or (3) the prioritization of contigs including complete putative AAV cap ORFs and further post hoc characterization of putative AAV cap ORF to confirm novelty separately, sequentially, or in combination.

Computing device 500 is intended to represent various forms of digital computers, such as laptops, desktops, workstations, personal digital assistants, servers, blade servers, mainframes, and other appropriate computers. Computing device 550 is intended to represent various forms of mobile devices, such as personal digital assistants, cellular telephones, smartphones, and other similar computing devices. Additionally, computing device 500 or 550 can include Universal Serial Bus (USB) flash drives. The USB flash drives can store operating systems and other applications. The USB flash drives can include input/output components, such as a wireless transmitter or USB connector that can be inserted into a USB port of another computing device. The components shown here, their connections and relationships, and their functions, are meant to be examples only, and are not meant to limit implementations of the methods and compositions described and/or claimed in this document.

Computing device 500 includes a processor 502, memory 504, a storage device 506, a high-speed interface 508 connecting to memory 504 and high-speed expansion ports 510, and a low speed interface 512 connecting to low speed bus 514 and storage device 506. Each of the components 502, 504, 506, 508, 510, and 512, are interconnected using various busses, and can be mounted on a common motherboard or in other manners as appropriate. The processor 502 can process instructions for execution within the computing device 500, including instructions stored in the memory 504 or on the storage device 506 to display graphical information for a GUI on an external input/output device, such as display 516 coupled to high speed interface 508. In other implementations, multiple processors and/or multiple buses can be used, as appropriate, along with multiple memories and types of memory. Also, multiple computing devices 500 can be connected, with each device providing portions of the necessary operations, e.g., as a server bank, a group of blade servers, or a multi-processor system.

The memory 504 stores information within the computing device 500. In one implementation, the memory 504 is a volatile memory unit or units. In another implementation, the memory 504 is a non-volatile memory unit or units. The memory 504 can also be another form of computer-readable medium, such as a magnetic or optical disk.

The storage device 506 is capable of providing mass storage for the computing device 500. In one implementation, the storage device 506 can be or contain a computer-readable medium, such as a floppy disk device, a hard disk device, an optical disk device, or a tape device, a flash memory or other similar solid-state memory device, or an array of devices, including devices in a storage area network or other configurations. A computer program product can be tangibly embodied in an information carrier. The computer program product can also contain instructions that, when executed, perform one or more methods, such as those described above. The information carrier is a computer- or machine-readable medium, such as the memory 504, the storage device 506, or memory on processor 502.

The high-speed controller 508 manages bandwidth-intensive operations for the computing device 500, while the low speed controller 512 manages lower bandwidth intensive operations. Such allocation of functions is only an example. In one implementation, the high-speed controller 508 is coupled to memory 504, display 516, e.g., through a graphics processor or accelerator, and to high-speed expansion ports 510, which can accept various expansion cards (not shown). In the implementation, low-speed controller 512 is coupled to storage device 506 and low-speed expansion port 514. The low-speed expansion port, which can include various communication ports, e.g., USB, Bluetooth, Ethernet, wireless Ethernet can be coupled to one or more input/output devices, such as a keyboard, a pointing device, microphone/speaker pair, a scanner, or a networking device such as a switch or router, e.g., through a network adapter. The computing device 500 can be implemented in a number of different forms, as shown in the figure. For example, it can be implemented as a standard server 520, or multiple times in a group of such servers. It can also be implemented as part of a rack server system 524. In addition, it can be implemented in a personal computer such as a laptop computer 522. Alternatively, components from computing device 500 can be combined with other components in a mobile device (not shown), such as device 550. Each of such devices can contain one or more of computing device 500, 550, and an entire system can be made up of multiple computing devices 500, 550 communicating with each other.

The computing device 500 can be implemented in a number of different forms, as shown in the figure. For example, it can be implemented as a standard server 520, or multiple times in a group of such servers. It can also be implemented as part of a rack server system 524. In addition, it can be implemented in a personal computer such as a laptop computer 522. Alternatively, components from computing device 500 can be combined with other components in a mobile device (not shown), such as device 550. Each of such devices can contain one or more of computing device 500, 550, and an entire system can be made up of multiple computing devices 500, 550 communicating with each other.

Computing device 550 includes a processor 552, memory 564, and an input/output device such as a display 554, a communication interface 566, and a transceiver 568, among other components. The device 550 can also be provided with a storage device, such as a micro-drive or other device, to provide additional storage. Each of the components 550, 552, 564, 554, 566, and 568, are interconnected using various buses, and several of the components can be mounted on a common motherboard or in other manners as appropriate.

The processor 552 can execute instructions within the computing device 550, including instructions stored in the memory 564. The processor can be implemented as a chipset of chips that include separate and multiple analog and digital processors. Additionally, the processor can be implemented using any of a number of architectures. For example, the processor 510 can be a CISC (Complex Instruction Set Computers) processor, a RISC (Reduced Instruction Set Computer) processor, or a MISC (Minimal Instruction Set Computer) processor. The processor can provide, for example, for coordination of the other components of the device 550, such as control of user interfaces, applications run by device 550, and wireless communication by device 550.

Processor 552 can communicate with a user through control interface 558 and display interface 556 coupled to a display 554. The display 554 can be, for example, a TFT (Thin-Film-Transistor Liquid Crystal Display) display or an OLED (Organic Light Emitting Diode) display, or other appropriate display technology. The display interface 556 can comprise appropriate circuitry for driving the display 554 to present graphical and other information to a user. The control interface 558 can receive commands from a user and convert them for submission to the processor 552. In addition, an external interface 562 can be provided in communication with processor 552, so as to enable near area communication of device 550 with other devices. External interface 562 can provide, for example, for wired communication in some implementations, or for wireless communication in other implementations, and multiple interfaces can also be used.

The memory 564 stores information within the computing device 550. The memory 564 can be implemented as one or more of a computer-readable medium or media, a volatile memory unit or units, or a non-volatile memory unit or units. Expansion memory 574 can also be provided and connected to device 550 through expansion interface 572, which can include, for example, a SIMM (Single In Line Memory Module) card interface. Such expansion memory 574 can provide extra storage space for device 550, or can also store applications or other information for device 550. Specifically, expansion memory 574 can include instructions to carry out or supplement the processes described above, and can also include secure information. Thus, for example, expansion memory 574 can be provided as a security module for device 550, and can be programmed with instructions that permit secure use of device 550. In addition, secure applications can be provided via the SIMM cards, along with additional information, such as placing identifying information on the SIMM card in a non-hackable manner.

The memory can include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 564, expansion memory 574, or memory on processor 552 that can be received, for example, over transceiver 568 or external interface 562.

Device 550 can communicate wirelessly through communication interface 566, which can include digital signal processing circuitry where necessary. Communication interface 566 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through radio-frequency transceiver 568. In addition, short-range communication can occur, such as using a Bluetooth, Wi-Fi, or other such transceiver (not shown). In addition, GPS (Global Positioning System) receiver module 570 can provide additional navigation- and location-related wireless data to device 550, which can be used as appropriate by applications running on device 550.

Device 550 can also communicate audibly using audio codec 560, which can receive spoken information from a user and convert it to usable digital information. Audio codec 560 can likewise generate audible sound for a user, such as through a speaker, e.g., in a handset of device 550. Such sound can include sound from voice telephone calls, can include recorded sound, e.g., voice messages, music files, etc. and can also include sound generated by applications operating on device 550.

The computing device 550 can be implemented in a number of different forms, as shown in the figure. For example, it can be implemented as a cellular telephone 580. It can also be implemented as part of a smartphone 582, personal digital assistant, or other similar mobile device.

Various implementations of the systems and methods described here can be realized in digital electronic circuitry, integrated circuitry, specially designed ASICs (application specific integrated circuits), computer hardware, firmware, software, and/or combinations of such implementations. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, e.g., magnetic discs, optical disks, memory, Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device, e.g., a CRT (cathode ray tube) or LCD (liquid crystal display) monitor for displaying information to the user and a keyboard and a pointing device, e.g., a mouse or a trackball by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The systems and techniques described here can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the systems and techniques described here, or any combination of such back end, middleware, or front end components. The components of the system can be interconnected by any form or medium of digital data communication, e.g., a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

A number of embodiments have been described. Nevertheless, it will be understood that various modifications can be made without departing from the spirit and scope of the invention. In addition, the logic flows depicted in the figures do not require the particular order shown, or sequential order, to achieve desirable results. In addition, other steps can be provided, or steps can be eliminated, from the described flows, and other components can be added to, or removed from, the described systems. Accordingly, other embodiments are within the scope of the following claims.

Embodiments of the disclosure and all of the functional operations described in this specification can be implemented in digital electronic circuitry, or in computer software, firmware, or hardware, including the structures disclosed in this specification and their structural equivalents, or in combinations of one or more of them. Embodiments of the methods and compositions can be implemented as one or more computer program products, e.g., one or more modules of computer program instructions encoded on a computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them. A propagated signal is an artificially generated signal, e.g., a machine-generated electrical, optical, or electromagnetic signal that is generated to encode information for transmission to suitable receiver apparatus.

A computer program (also known as a program, software, software application, script, or code) can be written in any form of programming language, including compiled or interpreted languages, and it can be deployed in any form, including as a stand alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a tablet computer, a mobile telephone, a personal digital assistant (PDA), a mobile audio player, a Global Positioning System (GPS) receiver, to name just a few. Computer readable media suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the disclosure can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the disclosure can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the methods, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

While this specification contains many specifics, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

In each instance where an HTML file is mentioned, other file types or formats may be substituted. For instance, an HTML file may be replaced by an XML, JSON, plain text, or other types of files. Moreover, where a table or hash table is mentioned, other data structures (such as spreadsheets, relational databases, or structured files) may be used.

### EXAMPLES

The invention is further described in the following examples, which do not limit the scope of the invention described in the claims.

### Example 1: Mining Metagenome Data for Novel AAV Capsids Identifies 44 Novel AAV Capsids and 3 Putative Undiscovered AAV Branches

In order to demonstrate the effectiveness of the computational pipeline disclosed herein including the sequence alignment-based module, the gene-based data processing module, and the further post hoc characterization of putative AAV cap ORFs to confirm novelty, the computational pipeline was performed on the NCBI Sequence Read Archive (SRA) dataset. First, using the sequence alignment-based module, SRA reads mapped to known viral genomes were quantified. The Serratus cloud computing infrastructure was used for this step. This step effectively detected presence/absence of known viruses in the SRA dataset based on the number of reads mapped. In this way, 5.8 million SRA samples were screened to extract approximately 9000 SRA samples containing at least some reads mapped to the Parvoviridae genomes.

Next, the gene-based data processing module was used to assemble contigs from the 9000 SRA samples. The Cenote-Taker2 program was used to filter out viral contigs, and filter on viral contig identity based on hallmark genes mapping as described above.

Finally, further post hoc characterization of putative AAV cap ORFs was performed to confirm novelty of contigs comprising putative AAV cap ORFs. In this step, cap ORFs were translated, contigs were filtered for complete cap ORFs, BLAST alignment sequence similarity search was performed against any known, patented AAV capsid sequences using NCBI database, and phylogenetic analysis was performed to place putative novel AAV cap ORFs among AAV isolates on a phylogenetic tree.

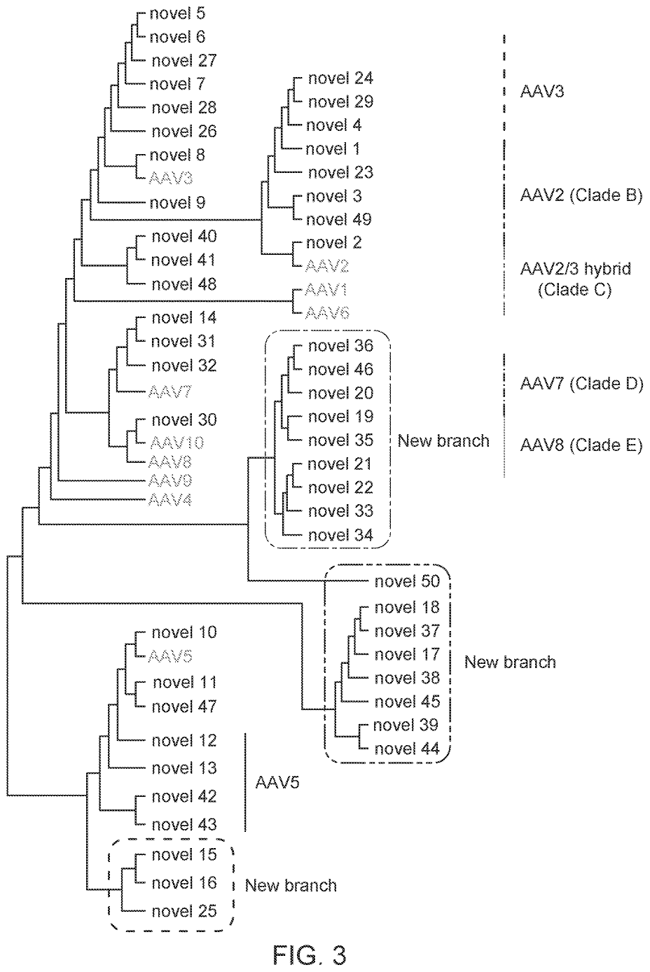

FIG. 3 shows a phylogenetic tree of previously known AAV clades. The computational pipeline performed on millions of SRA reads identified 44 novel AAV capsids, a subset of which form three putative undiscovered AAV branches, as indicated in FIG. 3.

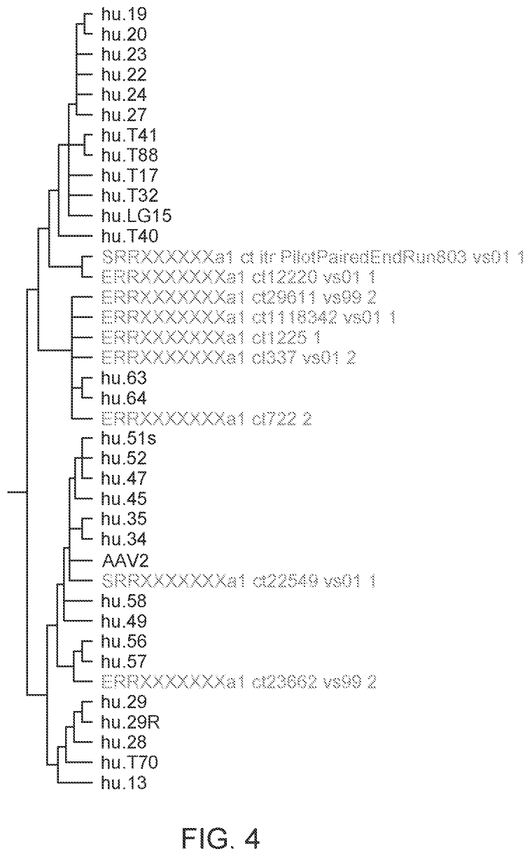

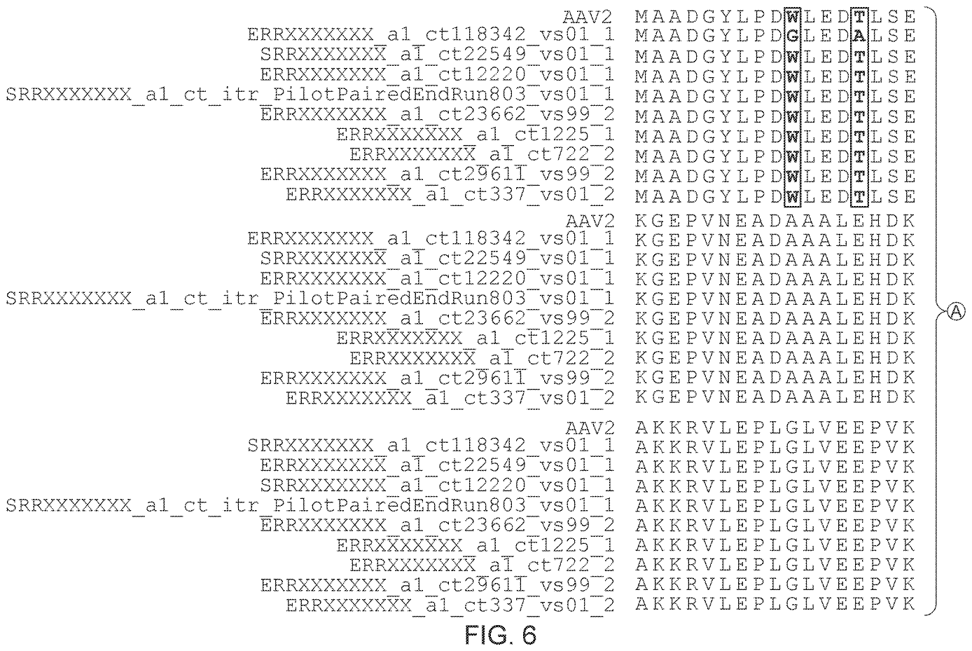

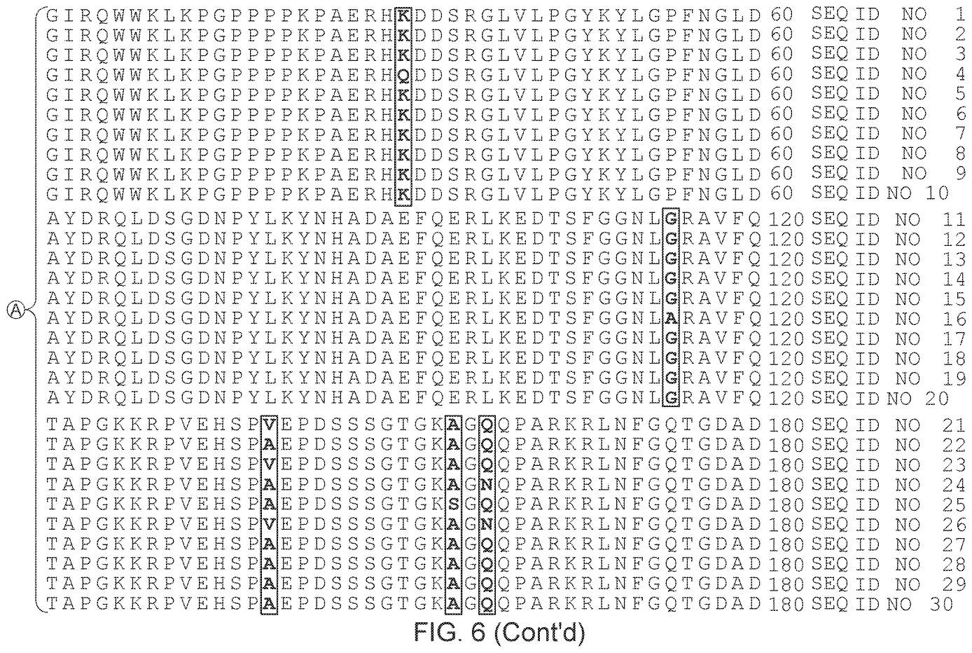

In further detail, FIG. 4 shows a subset of a phylogenetic tree of 163 natural isolates of AAV. As shown in FIG. 4, nine contigs close to AAV2 (Clade B) were identified by the computational pipeline as complete and novel. Multiple sequence alignment (MSA) of these novel AAV cap ORFs that are somewhat similar to AAV2shows that most of the amino acid differences between the novel AAV cap ORFs and AAV2 cap occur in variable regions, and are therefore not likely to be sequencing artifacts and may have functional impact on the AAV capsid. Variable regions within capsid protein sequences define AAV serotypes and dictate tropism by modifying the binding interactions between capsids and receptors that facilitate their cellular uptake. As shown in FIG. 6, highlighted by gray bars are amino acid residues of the nine novel AAV cap ORFs that are distinct compared to the AAV2 VP1 reference sequence. These results indicate that the computational pipeline disclosed herein can successfully identify polynucleotide sequences encoding novel AAV capsids in previously unexplored petabytes of metagenome data.

As a demonstrated by the putative novel AAV cap ORFs identified by the computational methods and systems disclosed herein, petabytes of publicly available metagenomic datasets can be mined to identify undiscovered putative AAV capsids from a variety of environmental samples. Using the computational pipeline disclosed herein, 44 putative novel AAV capsids have been identified in a short timeframe. It previously took over 60 years of research to assemble a list of 160 natural AAV isolates. The novel AAV capsids identified by the computation pipeline disclosed herein expands the current repertoire of AAV isolates available for clinically useful human gene therapies by almost 30%.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for identifying novel viral polypeptide sequences by screening polynucleotide sequencing data, the method comprising:
providing polynucleotide sequencing data comprising a plurality of sequencing reads;
aligning each of the plurality of sequencing reads to one or more reference sequences;
assembling, from the plurality of sequencing reads aligned to the one or more reference sequences, a plurality of sequence contigs;
selecting, based on the one or more reference sequences, one or more contigs comprising a viral open reading frame (ORF) sequence; and
identifying, based on a translated polypeptide sequence of the viral ORF, one or more novel viral polypeptide sequences.

2. The method of claim 1, wherein the one or more reference sequences are virus references sequences.

3. The method of claim 2, wherein the one or more virus reference sequences are Parvoviridae reference sequences.

4. The method of any one of claims 2-3, wherein the one or more virus reference sequences are adeno-associated virus (AAV) reference sequences.

5. The method of claim 4, wherein the AAV reference sequences are sequences encoding an AAV cap protein.

6. The method of any one of claims 1-5, wherein the polynucleotide sequencing data are metagenome data.

7. The method of any one of claims 1-6, wherein the plurality of sequencing reads are generated by next-generation sequencing (NGS).

8. The method of any one of claims 1-7, wherein the selecting step further comprises prescribing a minimum circular contig length.

9. The method of claim 8, wherein the minimum circular contig length is 4000 bp.

10. The method of any one of claims 1-9, wherein the selecting step further comprises prescribing a minimum linear contig length.

11. The method of claim 10, wherein the minimum linear contig length is 1500 bp.

12. The method of any one of claims 8-11, wherein the minimum circular contig length is 4000 bp and the minimum linear contig length is 1500 bp.

13. The method of any one of claims 1-12, wherein the identifying step further comprises prescribing a minimum ORF length.

14. The method of claim 13, wherein the minimum ORF length is 685 amino acids.

15. The method of any one of claims 1-14, wherein the identifying step further comprises BLAST alignment analysis.

16. The method of claim 15, wherein BLAST alignment analysis is performed against previously annotated natural AAV isolates and/or previously discovered Parvoviridae amino acid sequences.

17. A computer program product tangibly embodied on a computer-readable medium, comprising instructions, that when executed by one or more processors, are configured to:
receive polynucleotide sequencing data comprising a plurality of sequencing reads;
align each of the plurality of sequencing reads to one or more reference sequences;
assemble, from the plurality of sequencing reads aligned to the one or more reference sequences, a plurality of sequence contigs;
select, based on the one or more reference sequences, one or more contigs comprising a viral open reading frame (ORF) sequence;
identify, based on a translated polypeptide sequence of the viral ORF, one or more novel viral polypeptide sequences.

18. The computer program product of claim 17, wherein the one or more reference sequences are virus references sequences.

19. The computer program product of claim 18, wherein the one or more virus references are Parvoviridae sequences.

20. The computer program product of any one of claims 18-19, wherein the virus reference sequences are adeno-associated virus (AAV) reference sequences.

21. The computer program product of claim 20, wherein the AAV reference sequences are sequences encoding an AAV cap protein.

22. The computer program product of any one of claims 17-21, wherein the polynucleotide sequencing data are metagenome data.

23. The computer program product of any one of claims 17-22, wherein the plurality of sequencing reads are generated by next-generation sequencing (NGS).

24. The computer program product of any one of claims 17-23, wherein the selecting step further comprises prescribing a minimum circular contig length.

25. The computer program product of claim 24, wherein the minimum circular contig length is 4000 bp.

26. The computer program product of any one of claims 17-25, wherein the selecting step further comprises prescribing a minimum linear contig length.

27. The computer program product of claim 26, wherein the minimum linear contig length is 1500 bp.

28. The computer program product of any one of claims 24-27, wherein the minimum circular contig length is 4000 bp and the minimum linear contig length is 1500 bp.

29. The computer program product of any one of claims 17-28, wherein the identifying step further comprises prescribing a minimum ORF length.

30. The computer program product of claim 29, wherein the minimum ORF length is 685 amino acids.

31. The computer program product of any one of claims 17-30, wherein the identifying step further comprises BLAST alignment analysis.

32. The computer program product of claim 31, wherein BLAST alignment analysis is performed against previously annotated natural AAV isolates and/or previously discovered Parvoviridae amino acid sequences.
